(19) United States
(12) Patent Application Publication
(10) Pub. No.: US 2022/0313753 A1
(43) Pub. Date: Oct. 6, 2022

(54) BIFIDOBACTERIUM BIFIDUM BACTERIAL STRAIN, THE COMPOSITIONS THEREOF AND RELATED USES

(71) Applicant: SOFAR S.P.A., Trezzano, Rosa [MI] (IT)

(72) Inventors: Simone Domenico GUGLIELMETTI, Trezzano, Rosa [MI] (IT); Valentina TAVERNITI, Trezzano, Rosa [MI] (IT); Andrea BIFFI, Trezzano Rosa [MI] (IT); Walter FIORE, Trezzano Rosa [MI] (IT)

(21) Appl. No.: 17/620,648

(22) PCT Filed: Jul. 8, 2020
(86) PCT No.: PCT/IB2020/056423

§ 371 Date: Dec. 17, 2021

(30) Foreign Application Priority Data
Jul. 8, 2019 (IT) 102019000011193

(51) Int. Cl. A61K 35/745; A61P 1/00; A61K 9/00

(52) U.S. Cl. CPC A61K 35/745; A61P 1/00; A61K 9/0053; A61K 9/0031

(57) ABSTRACT

The present invention relates to a bacterial strain of Bifidobacterium bifidum MIMBb23 sg (=BbfIBSCM) DSM 32708, compositions comprising said bacterial strain and the use thereof in methods for the treatment of gastrointestinal disorders, in particular functional gastrointestinal disorders, such as, for example, irritable bowel syndrome (IBS) or inflammatory bowel diseases (IBDs).

The present invention relates to a bacterial strain belonging to the species Bifidobacterium bifidum identified as Bifidobacterium bifidum MIMBb23sg=BbfIBS01 (in short, B. bifidum MIMBb23sg DSM 32708, or B. bifidum BbfIBS01 DSM 32708). Furthermore, the present invention relates to compositions comprising a mixture comprising, or alternatively, consisting of said Bifidobacterium bifidum MIMBb23sg (=BbfIBS01) DSM 32708 and, optionally, said compositions comprise at least one food or pharmaceutical grade additive and/or excipient. Furthermore, the present invention relates to said Bifidobacterium bifidum MIMBb23sg (=BbfIBS01) DSM 32708 and said compositions for use in a method for the treatment of gastrointestinal diseases, disorders or symptoms, in particular functional or inflammatory gastrointestinal disorders, such as, for example, irritable bowel syndrome (IBS) or chronic inflammatory bowel diseases (IBDs).

Irritable bowel syndrome (IBS) belongs to the group of functional gastrointestinal disorders (FGIDs), a diagnostic category that can be defined based on the symptomatic presentation alone and characterised by the absence of an evident pathogenetic substrate. IBS is one of the most common gastrointestinal disorders, affecting about 15-20% of the population, where abdominal discomfort or pain is associated with changes in the intestinal habitat. Although reported in literature, the evident alterations of the lumen or of the gastrointestinal mucosa at the tissue, cell or molecular level are variable events and they have not been irrefutably identified in the IBS. Altered immune responses appear to be involved, but they cannot explain the symptoms entirely. Similarly, an alteration of the gut microbiota (i.e. a dysbiosis) contributes to pathophysiology, but no specific pathogen or pathobiont has yet been reliably related to the IBS.

Currently, the therapies available for the treatment of the IBS are aimed at the resolution of the pathogenetic events underlying the IBS. In the course of diarrhoea, the frequency of bowel movements can be decreased by reducing intake of short chain carbohydrates poorly absorbed in the small intestine (Fodmap) such as fructose, sorbitol and mannitol. It may be useful to combine kaolin-based preparations such as diosmectite with these solutions. Available in subjects with predominantly constipated small bowel transit and with presence of tympanites, are preparations with low concentrations of polyethylene glycols/mineral salts, to be taken daily. Furthermore, the use of linaclotide, a guanylate cyclase C receptor agonist, is also available in these subjects, who suffer from moderate-severe constipation. The use of anxiolytics (such as benzodiazepines) in the short periods when the patient recognises the anxiety thereof, is useful in reducing psychological participation in pain, with reduction thereof. Similarly, besides directly modulating pain without altering psychic function, the use of antidepressants such as tricyclics and selective serotonin reuptake inhibitors (SSRIs) is capable of improving sleep quality and decreasing the frequency of attacks. Other therapies instead aim at controlling pain; in this sense, some spasmolytics are particularly useful. Anticholinergic-antispasmodic drugs (antimuscarinic drugs), such as for example atropine, scopolamine, mebeverine, are used to reduce gastric secretion and intestinal motility. Similarly to the therapy of diverticular disease, tympani syndrome can be reduced by using poorly absorbable antibiotics, such as for example rifaximin, and probiotics that regulate gut flora.

However, the aforementioned treatments often do not allow a complete and lasting resolution of the disease and its symptoms.

The need therefore remains high to provide an effective solution for the treatment of gastrointestinal disorders, in particular functional or inflammatory gastrointestinal disorders, more in particular irritable bowel syndrome (IBS) or chronic inflammatory bowel diseases (IBDs).

Following an extensive research and development activity, the Applicant addresses and solves the present need by providing a new isolated bacterial strain belonging to the species Bifidobacterium bifidum, B. bifidum MIMBb23sg (=BbfIBS01) DSM 32708 or a derivative thereof (in short, bacterial strain of the invention), compositions comprising said bacterial strain or a derivative thereof (in short, composition/s of the invention) and the use thereof for the treatment of gastrointestinal disorders, preferably functional or inflammatory gastrointestinal disorders, more preferably irritable bowel syndrome (IBS) or chronic inflammatory bowel disease (IBD), as reported in the present description and in the claims.

The bacteria of the genus Bifidobacterium are Gram-positive bacteria, strictly anaerobic, belonging to the Actinobacteria phylum, which have been widely used as probiotics, i.e. “live microorganisms which, when administered in adequate amounts, confer a health benefit on the host”. Due to their resistance to stress during industrial production, the majority of the microbial biomasses of probiotics of the genus Bifidobacterium on the market consisting of B. animalis subsp. Lactis; however, scientific literature shows that other species of Bifidobacterium also exhibit promising probiotic properties. In particular, the species B. bifidum can be considered an optimal example of co-evolution between human host and microbes.

Specifically, the bacterial strain of the invention, B. bifidum MIMBb23sg DSM 32708, or a derivative thereof, and the compositions comprising said bacterial strain are effective in the treatment of gastrointestinal disorders, in particular with regard to functional gastrointestinal disorders, such as for example the IBS, given that—as illustrated in detail in the experimental part—the bacterial strain B. bifidum MIMBb23sg DSM 32708 modulates serotoninergic gene expression by inducing greater availability of serotonin in ileum and, therefore, inducing greater motility in the ileum and enhancement of peristalsis.

In addition, the bacterial strain B. bifidum MIMBb23sg (=BbfIBS01) DSM 32708 influences the expression of different genes involved in immune responses in the intestine, particularly in the ileum, making the anti-inflammatory/regulatory activity thereof in the intestine plausible.

Furthermore, the bacterial strain and the composition of the invention do not have significant adverse effects and they can be administered to all subjects, particularly to paediatric subjects and pregnant women.

Lastly, the composition of the invention is easy to prepare and economically advantageous.

These and other objects which will be clearer from the detailed description that follows, are achieved by the bacterial strain, by the compositions and by the mixtures of the present invention thanks to the technical characteristics claimed in the attached claims.

### BRIEF DESCRIPTION OF THE FIGURES

FIG. 1: genomic analysis with putative coding sequences of B. bifidum MIMBb23sg (=BbfIBS01) DSM 32708.

FIG. 2: PCR with strain-specific primers for the quantification of B. bifidum MIMBb23sg (=BbfIBS01) DSM 32708 and L. helveticus MIMLh5 in the ileum, cecum and colon.

FIG. 3: analysis of intra-sample diversity (a) evaluated by means of the Chao1 index, Faith's Phylogenetic diversity, Shannon and Inverse Simpson indices in the ileum, cecum and colon of mice treated with B. bifidum MIMBb23sg (=BbfIBS01) DSM 32708 and L. helveticus MIMLh5.

FIG. 4: analysis of inter-sample diversity (β) with the weighted and unweighted UniFrac algorithms in the ileum, cecum and colon for B. bifidum MIMBb23sg (=BbfIBS01) DSM 32708 and L. helveticus MIMLh5.

FIGS. 5a, 5b and 5c: cladograms in the ileum, cecum and colon for B. bifidum MIMBb23sg (=BbfIBS01) DSM 32708 and L. helveticus MIMLh5.

FIGS. 6a, 6b and 6c: cladograms of the LEfSe analysis for the identification of bacterial categories in the ileum, cecum and colon for mice treated with B. bifidum MIMBb23sg (=BbfIBS01) DSM 32708.

FIGS. 7a, 7b and 7c: analysis of the microbiota in the ileum, cecum and colon of mice treated with B. bifidum MIMBb23sg (=BbfIBS01) DSM 32708 and L. helveticus MIMLh5 by means of DESeq2 negative binomial distribution (OTU, operational taxonomic unit).

FIGS. 8 and 9a, 9b and 9c: RT-qPCR analysis of the expression of genes involved in the intestinal metabolism of serotonin in the ileum, cecum and colon of mice treated with B. bifidum MIMBb23sg (=BbfIBS01) DSM 32708 and L. helveticus MIMLh5.

FIGS. 10 and 11a, 11b and 11c: RT-qPCR analysis of gene expression of different cytokines, cyclooxygenase 2 (COX-2) and zonulin protein in the ileum, cecum and colon of mice treated with B. bifidum MIMBb23sg (=BbfIBS01) DSM 32708 and L. helveticus MIMLh5.

FIGS. 12 and 13: Effect of the strain B. bifidum MIMBb23sg DSM 32708 on IL-8 secretion in HT-29 cells respectively in the absence or presence of an inflammatory stimulus (Salmonella).

### DETAILED DESCRIPTION OF THE INVENTION

Forming an object of the present invention is a bacterial strain belonging to the species Bifidobacterium bifidum identified as Bifidobacterium bifidum MIMBb23sg (=BbfIBS01) DSM 32708 or a derivative thereof, wherein said bacterial strain was deposited, as provided for by the Budapest Treaty, at Deutsche Sammlung von Mikroorganismen und Zellkulturen GmbH (DSMZ) under deposit number DSM 32708 on 4 Dec. 2017 by Sofar S.p.A. (in short, B. bifidum MIMBb23sg DSM 32708, or B. bifidum BbfIBS01 DSM 32708 or strain of the invention). It is pointed out that it is still and only the same bacterial strain irrespective of the internal name MIMBb23sg=BbfIBS01, used by the Applicant.

The bacterial strain B. bifidum MIMBb23sg DSM 32708 is a bacterial strain isolated from the faeces of a healthy adult woman.

Preferably, the bacterial strain Bifidobacterium bifidum MIMBb23sg DSM 32708, or Bifidobacterium bifidum BbfIBS01 DSM 32708 is a probiotic bacterial strain. “Probiotics” are defined as live and viable microorganisms which when administered in adequate amounts confer health benefits on the host (FAO and WHO definition).

In the context of the present invention, the term “derivative” of the bacterial strain B. bifidum MIMBb23sg DSM 32708, or B. bifidum BbfIBS01 DSM 32708, is used to indicate the bacterial strain B. bifidum MIMBb23sg DSM 32708, or B. bifidum BbfIBS01 DSM 32708 tyndallized or inactivated by means of other methods such as gamma irradiation or sonication, or lysates or extracts of B. bifidum MIMBb23sg DSM 32708, or B. bifidum BbfIBS01 DSM 32708 (paraproboitics) or any derivative and/or component of B. bifidum MIMBb23sg DSM 32708, or B. bifidum BbIBS01 DSM 32708, preferably exopolysaccharide, parietal fraction, metabolites or metabolic bioproducts generated by B. bifidum MIMBb23sg DSM 32708, or B. bifidum BbfIBS01 DSM 32708 (postbiotics) and/or any other product derived from B. bifidum MIMBb23sg DSM 32708, or B. bifidum BbfIBS01 DSM 32708.

Forming an object of the present invention is a composition (in short, composition of the invention) comprising a mixture comprising or, alternatively, consisting of the bacterial strain Bifidobacterium bifidum MIMBb23sg DSM 32708, or BbfIBS01 DSM 32708, or a derivative thereof and, optionally, said composition comprises at least one food or pharmaceutical grade additive and/or excipient.

According to an aspect of the present disclosure, the composition of the invention comprises a single bacterial strain, such as the strain MIMBb23sg DSM 32708.

Preferably, the composition of the invention comprises the bacterial strain B. bifidum MIMBb23sg DSM 32708 at a concentration comprised in the range from 1×10⁶ CFU to 1×10¹² CFU, preferably from 1×10⁷ CFU to 1×10¹¹ CFU, more preferably from 1×10⁸ CFU to 1×10¹⁰ CFU, for example 1×10⁹ CFU, with respect to the daily intake (CFU: Colony Forming Unit).

In an embodiment, besides the bacterial strain B. bifidum MIMBb23sg DSM 32708, the mixture contained in the composition of the invention may comprise at least one further active component selected from the group comprising or, alternatively, consisting of other probiotic and/or paraprobiotic and/or postbiotic strains and/or lysed and/or tyndallized bacterial strains, enzymes, substances having direct or indirect antacid action, prebiotic substances, probiotic substances belonging to the families of yeasts and bacteria, immunostimulant substances, antidiarrheal substances, nutritional substances, vitamins of group B, C, D, E, organic and/or inorganic salts of magnesium, selenium, zinc, melatonin, valerian, passion flower, lemon balm, hawthorn, chamomile, hop plant, antioxidants, anti-radical agents.

The composition of the invention can be in solid form, such as tablet, chewable tablet, capsule, lozenge, granules, flakes or powder, in semi-solid form, such as soft-gel, gel, cream, balm, or in liquid form, such as solution, suspension, dispersion, emulsion or syrup.

The composition of the invention can be formulated for oral (or gastroenteric), sublingual (or buccal) or transmucosal, topical, rectal, cutaneous, vaginal use (or administration); it is advantageously formulated for oral use.

The composition of the invention optionally comprises said at least one food or pharmaceutical grade additive and/or excipient, i.e. a substance devoid of therapeutic activity suitable for pharmaceutical or food use. In the context of the present invention, the acceptable additives and/or excipients for pharmaceutical or food use comprise all the auxiliary substances known to the man skilled in the art for the preparation of compositions in solid, semi-solid or liquid form, such as, for example, diluents, solvents (including water, glycerine, ethyl alcohol), solubilisers, acidifiers, thickeners, sweeteners, flavour-enhancement agents, colouring agents, sweeteners, lubricants, surfactants, preservatives, pH stabilising buffers and mixtures thereof.

The composition of the invention, comprising the bacterial strain B. bifidum MIMBb23sg (=BbfIBS01) DSM 32708 in the various embodiments of the invention, may be a pharmaceutical composition (or Live Biotherapeutic Products), a medical device composition, a dietary supplement, a food (or novel food or medical food), a composition for a dietary supplement or food, a cosmetic composition, a composition for a food for special medical purposes (FSMP).

In the context of the present invention, the expression “medical device” is used in the meaning according to the Italian Legislative Decree no 46 dated 24 Feb. 1997 or according to the new Medical Device Regulation (EU) 2017/745 (MDR).

In the context of the present invention the term “novel food” is used in its meaning according to the EU Regulation 2015/2283 of 25 Nov. 2015.

Forming a further object of the present invention is the bacterial strain B. bifidum MIMBb23sg (=BbfIBS01) DSM 32708, or a derivative thereof, and the composition of the invention, comprising or, alternatively, consisting of B. bifidum MIMBb23sg (=BbfIBS01) DSM 32708, for use as a medicament.

In an embodiment, the bacterial strain B. bifidum MIMBb23sg (=BbfIBS01) DSM 32708, or a derivative thereof, and the composition of the invention, which comprises the mixture comprising or, alternatively, consists of B. bifidum MIMBb23sg (=BbfIBS01) DSM 32708, are for use in a method for preventive and/or curative treatment of gastrointestinal diseases, disorders or symptoms in a subject in need, preferably functional gastrointestinal disorders, such as irritable bowel syndrome (IBS), dyspepsia, pyrosis, disorders of the oesophagus, stomach and duodenum disorders, small intestinal bacterial overgrowth (SIBO), disorders with sub-inflammatory conditions, for example in the elderly or in the celiac disease, in the diverticular disease.

In a further embodiment, the bacterial strain B. bifidum MIMBb23sg (=BbfIBS01) DSM 32708, or a derivative thereof, and the composition of the invention, which comprises the mixture comprising, or alternatively, consisting of B. bifidum MIMBb23sg (=BbfIBS01) DSM 32708, are for use in a method for preventive and/or curative treatment of inflammatory gastrointestinal diseases, disorders or symptoms in a subject in need, such as Helicobacter pylori, peptic or gastric ulcer, duodenal ulcer, chronic inflammatory bowel diseases, such as Crohn's disease and ulcerative colitis, microscopic colitis, celiac disease, diverticular disease and diverticulitis.

The efficacy of the strain B. bifidum MIMBb23sg DSM 32708 in treating inflammatory gastrointestinal disorders or symptoms is demonstrated by its ability to both increase the levels of the IL-10 (T-re, anti-inflammatory) cytokine expression in vivo in mice and to reduce the levels of the IL-8 (T-re, pro-inflammatory) cytokine expression in vitro.

In particular, the bacterial strain B. bifidum MIMBb23sg DSM 32708, or a derivative thereof, and the composition of the invention, which comprises the mixture comprising, or alternatively, consisting of B. bifidum MIMBb23sg DSM 32708, are for use as immunomodulants capable of modulating the immune system, in particular, by upregulating the IL-10 (T-re, anti-inflammatory) cytokine of the subject to whom they are administered. Therefore, the bacterial strain, or a derivative thereof, and the compositions of the present invention have a valid application for the preventive or curative treatment of diseases related with alterations of the immune system, in particular autoimmune diseases and allergies (or allergic diseases), skin diseases, such as acne, atopic dermatitis.

The bacterial strain of the present invention can also be used in a method for the treatment of central nervous system diseases, preferably anxiety and/or depression or related symptoms, since said strain in the intestine increases serotonin production and reduces reuptake.

Forming an object of the present invention is a method for the preventive or curative treatment of gastrointestinal diseases, disorders or symptoms in particular functional gastrointestinal disorders, preferably the IBS, which provides for the administration of the bacterial strain B. bifidum MIMBb23sg (=BbfIBS01) DSM 32708 or of the composition of the invention in a subject in need.

In the context of the present invention, the expression “subjects” is used to indicate human subjects or animal subjects (e.g. pets, such as dogs or cats or other mammals). Preferably, the compositions of the invention are for use in treatment methods for human subjects.

In the context of the present invention, the expression “treatment method” is used to indicate an intervention on a subject in need, comprising the administration of the bacterial strain or of a composition of the invention to the subject at a therapeutically effective amount, with the aim of eliminating, reducing/decreasing or preventing a disease or ailment and the symptoms or disorders thereof.

The expression “therapeutically effective amount” refers to the amount of composition and/or bacterial strain that elicits the biological or medicinal response in a tissue, system, mammal, or human being that is sought and defined by an individual, researcher, veterinarian, physician, or other clinician or health worker.

Embodiments (FRn) of the present invention are reported hereinafter.

FR1. A bacterial strain belonging to the species Bifidobacterium bifidum identified as Bifidobacterium bifidum MIMBb23sg=BbfIBS01, wherein said bacterial strain was deposited at Deutsche Sammlung von Mikroorganismen und Zellkulturen GmbH (DSMZ) under deposit number DSM 32708 on 4 Dec. 2017 by Sofar S.p.A.

FR2. The bacterial strain according to FR1 for use as a medicament.

FR3. The bacterial strain according to FR1 or FR2 for use in a method for the preventive or curative treatment of gastrointestinal diseases, disorders or symptoms preferably functional gastrointestinal disorders or inflammatory gastrointestinal disorders.

FR4. The bacterial strain for use according to FR3, wherein said strain is for use in a method for preventive and/or curative treatment of functional gastrointestinal disorders selected from: irritable bowel syndrome (IBS), dyspepsia, pyrosis, oesophagus, stomach and duodenum disorders, small intestinal bacterial overgrowth (SIBO), disorders with sub-inflammatory conditions, preferably wherein said sub-inflammatory disorders are manifested in an elderly or in a celiac subject, or in a subject suffering from diverticular disease.

FR5. The bacterial strain for use according to FR3, wherein said strain is for use in a method for preventive and/or curative treatment of inflammatory gastrointestinal disorders or symptoms selected from: Crohn's disease, ulcerative colitis, microscopic colitis, Helicobacter pylori, peptic or gastric ulcer, duodenal ulcer, celiac disease, diverticular disease and diverticulitis.

FR6. A composition comprising:

a mixture comprising or, alternatively, consisting of the bacterial strain Bifidobacterium bifidum MIMBb23sg=BbfIBS01 DSM 32708 according to FR1,

and, optionally, said composition comprises at least one food grade or pharmaceutical additive and/or excipient.

FR7. The composition according to FR6, wherein said mixture comprises the bacterial strain B. bifidum MIMBb23sg=BbfIBS01 DSM 32708 at a concentration comprised in the range from 1×10⁶ CFU to 1×10¹² CFU, preferably from 1×10⁷ CFU to 1×10¹¹ CFU, more preferably from 1×10⁸ CFU to 1×10¹⁰ CFU, with respect to a daily intake.

FR8. The composition according to any one of FR6 or FR7 for use as a medicament.

FR9. The composition according to any one of FR6 or FR7 for use in a method for the preventive or curative treatment of gastrointestinal diseases, disorders or symptoms preferably functional gastrointestinal disorders or inflammatory gastrointestinal disorders.

FR10. The composition for use according to FR9, wherein said composition is for use in a method for preventive and/or curative treatment of functional gastrointestinal disorders selected from: irritable bowel syndrome (IBS), dyspepsia, pyrosis, oesophagus, stomach and duodenum disorders, small intestinal bacterial overgrowth (SIBO), disorders with sub-inflammatory conditions, in an elderly or in a celiac subject, or in a subject suffering from diverticular disease.

FR11. The composition for use according to FR9, wherein said composition is for use in a method for preventive and/or curative treatment of inflammatory gastrointestinal disorders or symptoms selected from: Crohn's disease, ulcerative colitis, microscopic colitis, Helicobacter pylori, peptic or gastric ulcer, duodenal ulcer, celiac disease, diverticular disease and diverticulitis.

FR12. The bacterial strain for use according to FR2, or, alternatively, the composition for use according to FR8, wherein said bacterial strain or said composition is for use in a method for the preventive and/or curative treatment of anxiety and/or depression or related symptoms.

### EXPERIMENTAL PART

In order to contribute to the understanding of the action mechanisms of B. bifidum MIMBb23sg (=BbfIBS01) DSM 32708 in the host intestine, the impact of taking B. bifidum MIMBb23sg (=BbfIBS01) DSM 32708 in vivo on microbiota and on the gene expression of the host in the various intestinal regions was studied in a murine model. The same experiments were also conducted with Lactobacillus helveticus MIMLh5, selected as the reference bacterium being a probiotic strain with demonstrated ability to interact with the host which was isolated from a dairy environment.

The experimental part reported below shows that although both strains can affect the composition of the gut microbiota, the human isolate B. bifidum MIMBb23sg (=BbfIBS01) DSM 32708 evidently has a greater ability to influence the host mucosal gene expression than the dairy isolate L. helveticus MIMLh5.

Materials and Methods

(I) Bacterial Strains, Preparation, and Growth Conditions.

L. helveticus MIMLh5 and B. bifidum MIMBb23sg (=BbfIBS01) DSM 32708 were cultured in the Man-Rogosa-Sharpe (MRS) culture medium (Difco Laboratories Inc., Detroit, Mich., USA), with the addition of 0.05% of L-cysteine hydrochloride (Sigma-Aldrich) for B. bifidum MIMBb23sg (cMRS). Bacterial strains were inoculated from stocks frozen in glycerol and sub-cultured twice in MRS or cMRS using a 1:100 inoculum. The incubation temperature was 37° C. under aerobic conditions as concerns L. helveticus MIMLh5, while B. bifidum MIMBb23sg DSM 32708 was cultured in anaerobiosis jars using Anaercocult® strips (Merck Millipore). For the preparation of fresh cultures for use in in vivo experiments, bacterial cells derived from an overnight grown culture (18 hrs of growth) were collected, washed twice with sterile PBS and then resuspended in PBS at the concentration of 1×10⁹ cells ml⁻¹, using the Neubauer improved counting chamber. Bacterial cells were prepared fresh for each day of treatment of the mice.

(II) Treatment of Mice with Probiotic Strains.

Two-month-old female C57BL/6 mice (Charles River, Lecco, Italy) were housed in a conventional, non-sterile, pathogen-free stable. After one week of adaptation, the mice were separated into cages, in 3 groups of 5 mice each. Each group received the suspensions or carrier (PBS, phosphate buffered saline) once a day for 5 days. Bacterial cells of L. helveticus MIMLh5 or B. bifidum MIMBb23sg (=BbfIBS01) DSM 32708 were administered by means of oral gastric probe as 200 μl suspension. Sterile PBS, used as a carrier in the control groups, was administered using the same procedure. Mice were sacrificed 4 hours after the last administration. After sacrifice, two biopsy samples of distal ileum, cecum and proximal colon were collected from each mouse. One of the two biopsy samples collected from each intestinal tract was preserved at −80° C. for DNA extraction (microbiota analysis), while the other was removed from the intestinal contents by washing the tissue with a syringe containing sterile PBS, immediately transferred to a test tube containing 1 mL RNAlater (Qiagen) and preserved at −80° C. for RNA extraction (gene expression analysis). All these steps were carried out by maintaining mice and tissues on cooled trays.

(III) Isolation of Nucleic Acids from Intestinal Biopsies.

DNA was obtained from murine biopsies using the PowerFecal® DNA Isolation Kit (MO BIO Laboratories). The homogenisation of murine biopsies was performed using a Precellys bead beater (3×30″ at 6800 rpm; Advanced Biotech Italia s.r.l., Seveso, Italy). Subsequently, DNA isolation was conducted according to the manufacturer's instructions. For RNA isolation, washed biopsies preserved at −80° C. were thawed on ice for subsequent RNA extraction. The biopsies were then immediately resuspended in Qiazol (Qiagen) and homogenized by means of IKA T10 basic Ultraturrax (30,000 rpm for 30 s). The RNA extraction steps were then performed using the RNeasy Lipid Tissue Mini Kit (Qiagen), according to the manufacturer's instructions. The concentration and purity of nucleic acids were determined using the Take3 Micro-Volume (BioTek Instrument).

(IV) Microbiota Analysis.

In order to define the structure of the bacterial community in the various intestinal regions, the total DNA extracted from intestinal biopsies, as described above, was used to obtain profiling from the analysis of the gene region encoding the 16S ribosomal RNA subunit with the Illumina MiSeq System at the Center for Life—Nanoscience, Istituto Italiano di Tecnologia (Rome, Italy). In short, a DNA region comprising the V3 and V4 regions of the 16S rRNA gene was amplified with the primer pair described in Klindworth et al. (Klindworth et al., 2013). The sequence readings were analysed using Quantitative Insights Into Microbial Ecology (QIIME) software version 1.7.0 using GreenGene (dd_13_5) as the reference taxonomic database. The bacterial abundance of each sample was analysed at the operating taxonomic unit (OTU) level. The gene profile sequences of 16S ribosomal RNA were deposited at the European Nucleotide Archive (ENA) of the European Bioinformatics Institute under access code PRJEB00000.

(V) RNA Preparation and Reverse Transcription

After extraction, RNA integrity was verified by loading 100 ng of RNA onto 1% agarose gel under non-denaturing conditions. Thereafter, DNA removal was carried out by means of DNase I (Sigma-Aldrich) according to the manufacturer's protocol. In short, 8 μl of RNA were incubated with 1 μl of DNase I for 30 minutes at room temperature; thereafter, DNase was inactivated by adding 1 μl of Stop solution (Sigma-Aldrich) and by incubating at 70° C. for 10 minutes. RNA was re-quantified after DNA removal. One microgram of total RNA was then subjected to reverse transcription with the iScript Select cDNA Synthesis Kit (Bio-Rad Italia, Segrate, Italy) using the following thermal cycle: 5 minutes at 25° C., 30 minutes at 42° C., and 5 minutes at 85° C. The expression levels of the genes of interest were determined by quantitative PCR on the complementary DNA (cDNA) obtained by means of reverse transcription (RT-qPCR), using SYBR Green technology, using SsoFast EvaGreen Supermix (Bio-Rad Italia, Segrate, Italy) on a Bio-Rad CFX96 machine according to the manufacturer's instructions. Primers, if not derived and adapted from the literature, were designed using the Primer3 Tool and verified using an OligoAnalyzer 3.1 Tool and for specificity using Nucleotide BLAST. The primers used are listed in Table 1.

TABLE 1

| Name of the primer | Sequence |
|---|---|
| GAPDH F | ATGACCACAGTCCATGCCATC |
| GAPDH R | GGTCCTCAGTGTAGCCCAAG |
| SERT F | CAA AACCAAGAACCAAGAG |
| SERT R | CATAGCCAATGACAGACAG |
| 5-HTR3 F | GTGATAAGCCTCGCTGAGACC |
| 5-HTR3 R | CGCATCTCATCCCGCTTCT |
| 5-HTR4 F | GATGCCCTTTGGTGCCAT |
| 5-HTR4 R | CAGCAGATGGCGTAATACCTG |
| TPH-1 F | ATGAGAGAATTTGCCAAGACC |
| TPH-1 R | CGTGAACTATATTTCCCTCAG |
| ZONU F | GCTATGTGGATTGGTT |
| ZONU R | TCCATAGAGCGATGAT |
| BopA F: | GTGTTCCCACCAACTACA |
| BopA R: | GATCTGGTCGTAACCAGT |
| HELV F: | AAACGGGCATTTTGTGGGCTAT |
| HELV R: | GAAGCTTAAGGTTGAAGATGCC |
| 357 F: | CCTACGGGAGGCAGCAG |
| 907 R: | CCGTCAATTCCTTTGAGTTT |

Gradient and efficiency analyses were carried out to select the most suitable temperatures and concentrations for primer annealing. Thereafter, amplifications of the selected target genes were conducted in a total volume of 15 μl containing 2x SsoFast EvaGreen Supermix, primer forward and reverse (concentration of 300 μM for ZONU, 5HTR3 and 5HTR4; concentration of 500 μm for other primer pairs), ultra-pure sterile water, and cDNA (15 ng in reaction). The parameters of the qPCR cycle were as follows: 3 minutes at 95° C., followed by 44 cycles of 10 seconds at 95° C., 30 seconds at 58° C. and 5 seconds at 72° C. using the Bio-Rad CFX96 instrument. An annealing temperature of 55.5° C. was used for THP1 and ZONU primers. The amplification reactions were carried out in duplicate, and controls were carried out for possible contamination by genomic DNA. The amplifications were normalized with respect to the expression of the gene encoding glyceraldehyde 3-phosphate dehydrogenase, which proved to be the most stable reference gene in the preliminary comparative experiments with respect to the 18S and beta-actin genes (data not shown). Relative transcription levels were calculated using the 2^{(-ΔΔCT)} method. The specific amplification was verified by analysing melting curves and confirmed by analysing the amplification products on agarose gels.

(VI) Statistical Analysis.

Statistical calculations were carried out using the GraphPad Prism 5 software program. The significance of the results was analysed with the Mann-Whitney unpaired test with two-tailed distribution. A p<0.05 value was considered significant.

Differences in microbiota composition between the groups of mice were determined using the Wald test after normalization of counts of DESeq2 readings. Differences in microbial composition between the groups were also defined with the LDA Effect Size (LEfSe) analysis. Specifically, the Kruskal-Wallis test and the Wilcoxon test with paired data were carried out during the LEfSe analysis, considering a p<0.05 value for both statistical methods as significant.

(VII) Declaration of Ethics.

All the experiments were accepted by the Ethics Committee of the University of Milan (protocol no 3/2013).

Results

(VIII) Bifidobacterium bifidum MIMBb23sg DSM 32708 is a Strain Characteristic of its Species.

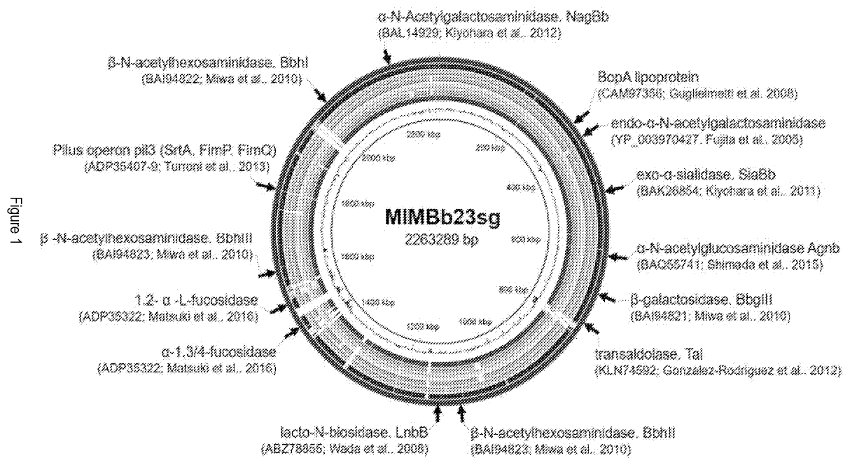

In this study, B. bifidum MIMBb23sg DSM 32708, which is a bacterial strain isolated from the faeces of a healthy adult woman, was evaluated. To preliminarily define the probiotic potential of B. bifidum MIMBb23sg DSM 32708, a genomic sequence draft consisting of 9 contigs for a total of 2,263,289 bp, with a guanine and cytosine content of 62.6%, which is consistent with that of other B. bifidum genomes, was generated (Guglielmetti et al, 2014b). Comparative genomic analysis showed that more than 90% of the putative coding sequences of B. bifidum MIMBb23sg DSM 32708 share a high sequence similarity with similar regions of other genomes of B. bifidum available at the GenBank. It has been observed above all that B. bifidum MIMBb23sg DSM 32708 has the genes that, in other strains of B. bifidum, have shown an involvement in the transport and metabolism of carbohydrates derived from the host and from the diet, such as mucin and oligosaccharides of breast milk (FIG. 1). Genes whose presumed protein products are involved in the interaction with the host intestinal mucosa, such as pili, lipoprotein Bop A, and transaldolase (Tal), were also observed.

The genome analysis of B. bifidum MIMBb23sg DSM 32708 did not reveal the presence of genes known for antibiotic resistance, in accordance with the antibiotic resistance profile that was determined with the microdilution analysis recommended by the International Organization for Standardization (ISO, 2010). In fact, the minimum inhibitory concentrations (MIC) of B. bifidum MIMBb23sg DSM 32708 did not exceed the EFSA limit values provided for bifidobacteria (EFSA, 2012) for any antibiotic tested (Table 2).

Overall, these results indicate that B. bifidum MIMBb23sg DSM 32708 possesses the important genetic characteristics that confirm the ability to colonise the intestinal tract of the host; B. bifidum MIMBb23sg DSM 32708 is also suitable for food/probiotic applications since it does not report any acquired antibiotic resistance.

In light of these results, B. bifidum MIMBb23sg DSM 32708 was used in the in vivo trials on mice.

TABLE 2

| | MIC of MIMBb23sg | EFSA limit values |
|---|---|---|
| antibiotic | (μg/ml) | (μg/ml) |
| ampicillin | 0.25 | 2 |
| vancomycin | 0.5 | 2 |
| gentamicin | 32 | 64 |
| kanamycin | 128 | n.r. |
| streptomycin | 128 | 128 |
| erythromycin | 0.25 | 1 |
| clindamycin | 0.25 | 1 |
| tetracycline | 1 | 8 |
| chloramphenicol | 2 | 4 |

(IX) B. bifidum MIMBb23sg DSM 32708 and L. helveticus MIMLh5 Modify the Bacterial Load in Different Intestinal Sites of Mice.

Quantitative PCR with strain-specific primers was used to quantify B. bifidum MIMBb23sg DSM 32708 and L. helveticus MIMLh5 in the ileum, cecum and colon of mice treated with bacterial cells or PBS by means of gastric probe, once per day for 5 days.

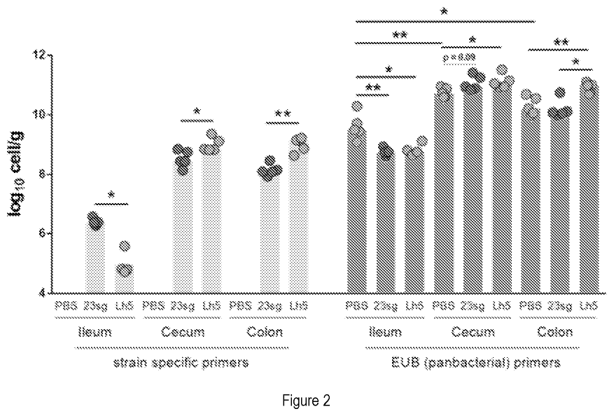

As expected, qPCR was negative in mouse samples treated with PBS by means of gastric probe. On the contrary, 6.4, 8.5 and 8.1 log₁₀ cells/g in the ileum, cecum, and colon of mice treated with B. bifidum MIMBb23sg DSM 32708 by means of gastric probe and 5.0, 8.9 and 9, respectively, 5.0, 8.9 and 9.2 log₁₀ cells/g in the ileum, cecum, and colon of mice treated with L. helveticus MIMLh5 by means of gastric probe were quantified. As a result, the number of both strains was significantly higher in the cecum and the colon than in the ileum (FIG. 2); in addition, strain B. bifidum MIMBb23sg DSM 32708 was significantly more abundant with L. helveticus MIMLh5 in the ileum, and less abundant in the cecum and in the colon. Subsequently, a qPCR with panbacterial primers targeting the 16S rRNA gene to quantify the total bacterial cells in the same murine intestinal samples used for the quantification of B. bifidum MIMBb23sg DSM 32708 and L. helveticus MIMLh5 was carried out. As expected, a significant concentration of bacterial cells in the cecum and in the colon (10.9 and 10.1 log₁₀ cells/g, respectively) compared to the ileum (9.0 log₁₀ cells/g) was observed; the difference between the bacterial concentration in the cecum and in the colon was also statistically significant (FIG. 2). Above all, a significant reduction in bacterial concentration was observed in the ileum of mice treated with B. bifidum MIMBb23sg DSM 32708 by means of gastric probe and L. helveticus MIMLh5 compared to mice treated with PBS; in contrast, the administration of L. helveticus MIMLh5 induced a significant increase in the bacterial load in the cecum compared to treatment with PBS and in the colon compared to both PBS and B. bifidum MIMBb23sg DSM 32708 (FIG. 2).

Overall, these data demonstrate that B. bifidum MIMBb23sg DSM 32708 and L. helveticus MIMLh5 predominantly colonised the cecum and the colon and led to a reduction in the number of total bacteria in the ileum. These results also indicate that B. bifidum MIMBb23sg DSM 32708 colonised ileum to a greater extent than L. helveticus MIMLh5, which, on the other hand, was more abundant than B. bifidum MIMBb23sg DSM 32708 in the cecum and colon.

(X) B. bifidum MIMBb23sg DSM 32708 and L. helveticus MIMLh5 Modulate the Microbiota of the Ileum, Cecum and Colon Differently.

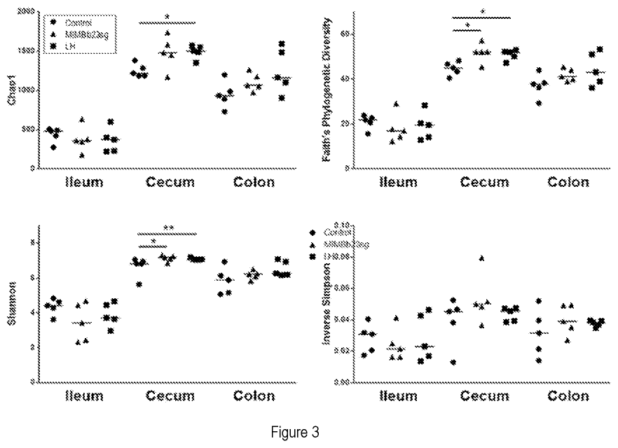

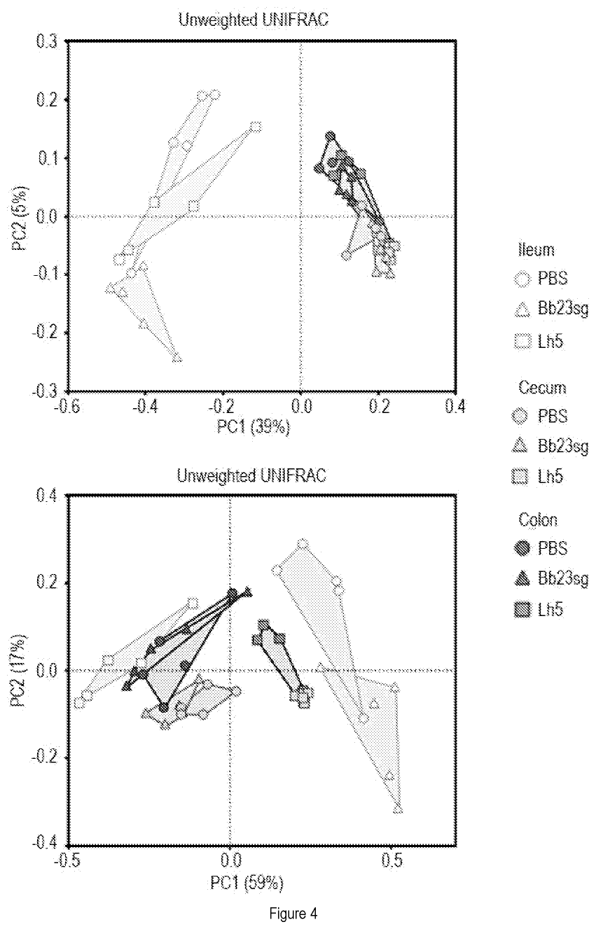

In order to better understand the impact of taking B. bifidum MIMBb23sg DSM 32708 and L. helveticus MIMLh5 on the overall composition of the intestinal microbiota, profiling was carried out by analysing the 16S rRNA gene on the same intestinal samples used in the qPCR experiments. The intra-sample diversity (α-diversity) analysis revealed an increase in Chao1 indices (for L. helveticus MIMLh5 only), Faith's Phylogenetic diversity and Shannon's diversity index in mice treated with bacteria through gavage, exclusively in the cecum (FIG. 3). Subsequently, inter-sample diversity (R-diversity) was analysed using the UniFrac algorithms; the unweighted UniFrac showed that the ileum microbiota differs from the cecum and colon microbiota (FIG. 4); it was also observed that, according to the unweighted UniFrac, B. bifidum MIMBb23sg DSM 32708 had a greater influence on the ileum microbiota with respect to the strain L. helveticus MIMLh5. In contrast, the weighted UniFrac showed that L. helveticus MIMLh5 modified the microbiota composition of the three intestinal sites to a greater extent with respect to B. bifidum MIMBb23sg DSM 32708 (FIG. 4).

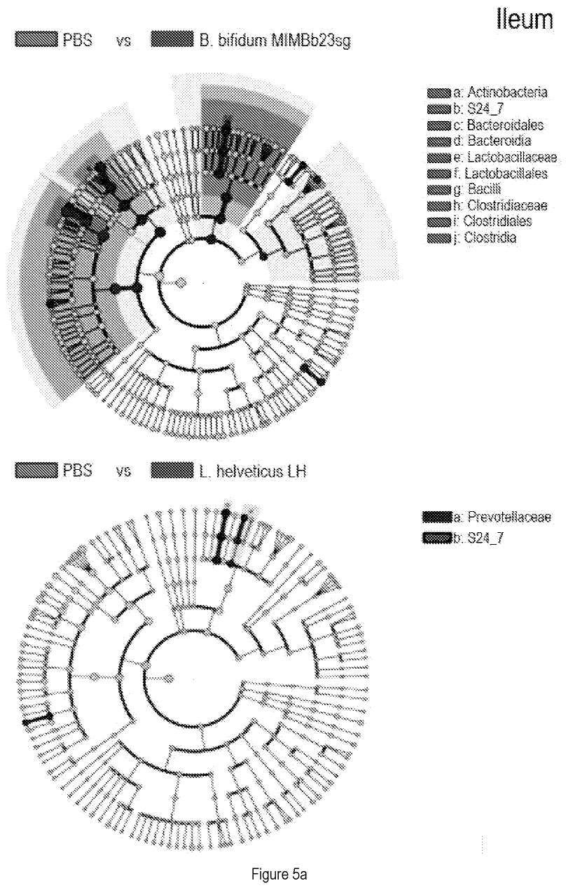

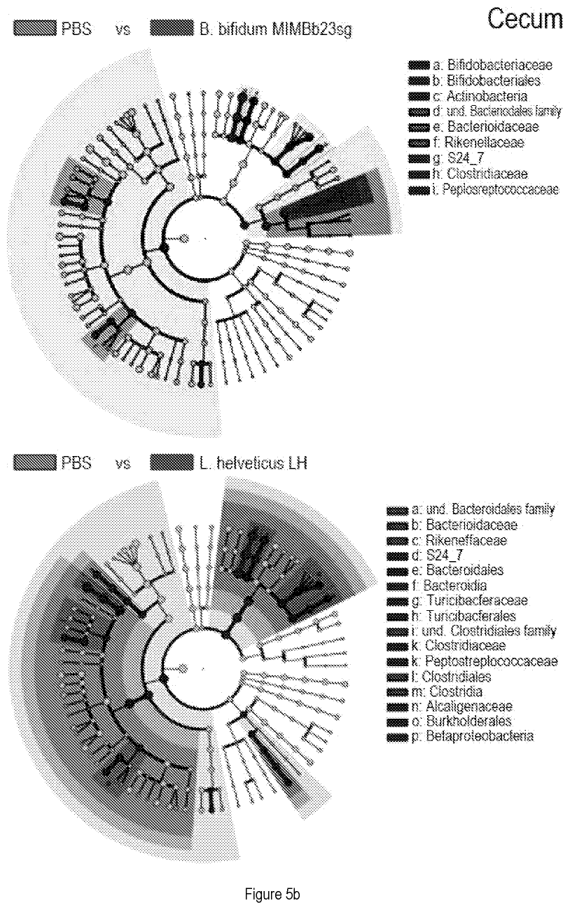

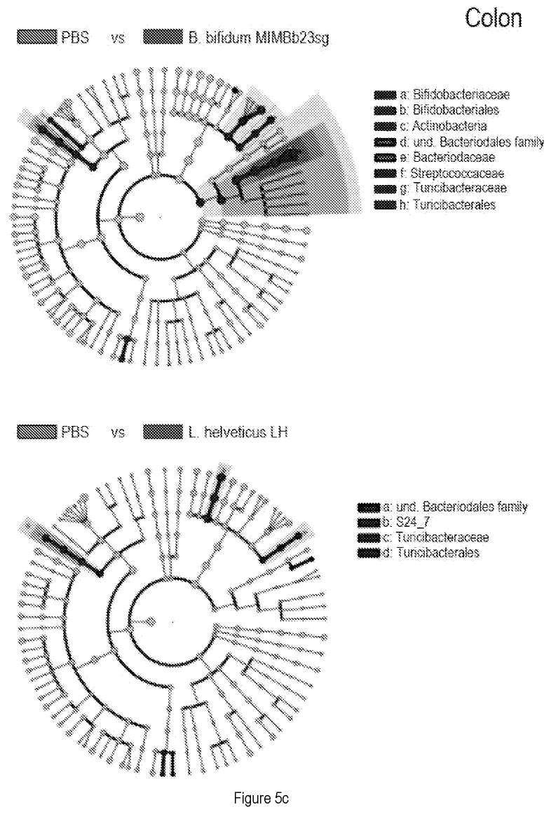

Thereafter, the LEfSe analysis was used to compare the microbiota of mice treated with PBS and bacteria in the three different intestinal sites. The resulting cladograms show that, in the ileum, B. bifidum MIMBb23sg DSM 32708 modified the abundance of more categories with respect to the strain L. helveticus MIMLh5 (FIG. 5a), while L. helveticus MIMLh5 influenced the microbiota of the cecum to a greater extent with respect to B. bifidum MIMBb23sg DSM 32708 (FIG. 5b). In contrast, the microbiota of the colon was affected in a similar manner (FIG. 5c). In particular, B. bifidum MIMBb23sg DSM 32708 increased the relative abundance of the Clostridiales categories, reducing Bacteroidales (in particular category S24-7) and Lactobacillales in ileum, while the strain L. helveticus MIMLh5 reduced the Clostridiales categories and increased Bacteroidales in the cecum.

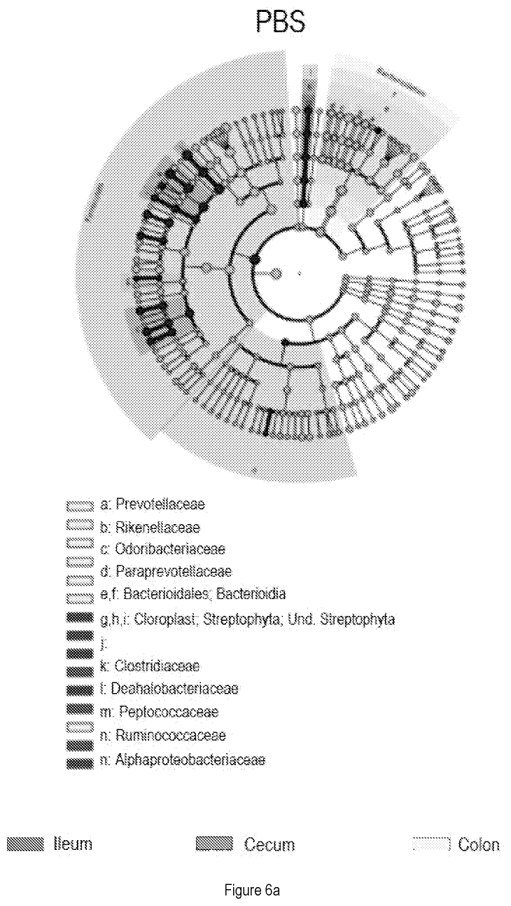

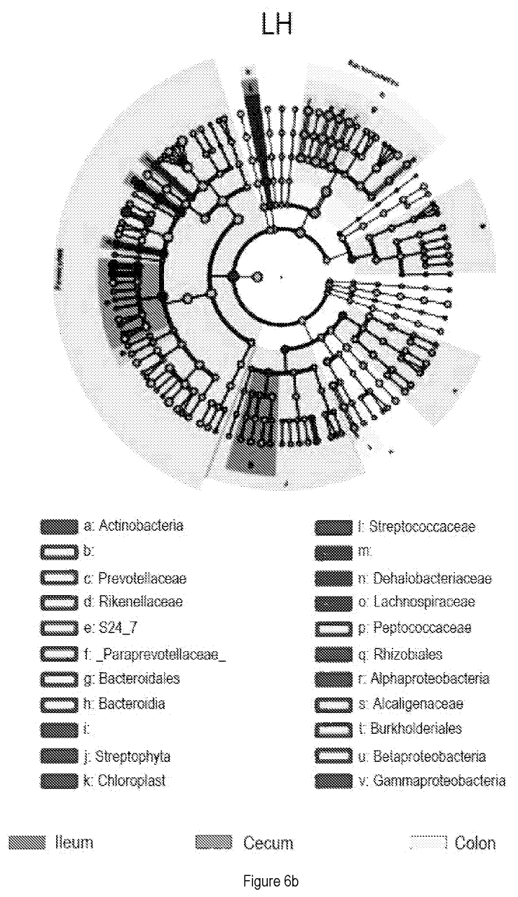

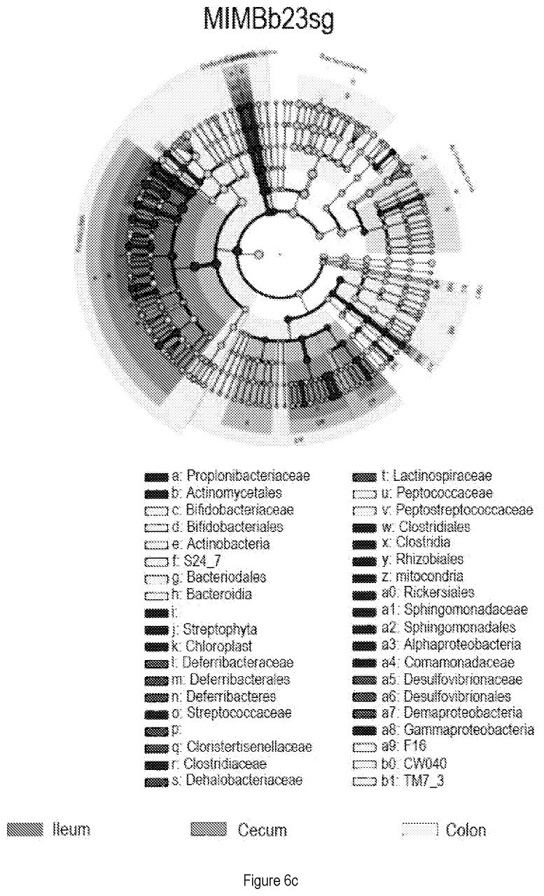

LEfSe analysis was also used to identify significantly different bacterial categories among intestinal regions within the same group of mice. The cladograms obtained demonstrated that mice treated with B. bifidum MIMBb23sg DSM 32708 exhibited an evident numerical increase in the categories, whose relative abundance was significantly different between the ileum and the colon (FIGS. 6a, 6b and 6c). Subsequently, the method of the DESeq2 negative binomial distribution was adopted in the analysis of the microbiota data to infer the relative differential abundance at the OTU (operational taxonomic unit) level between mice treated with PBS and bacteria by means of gastric probe. There were evident differences between the three intestinal regions and between the two bacterial strains.

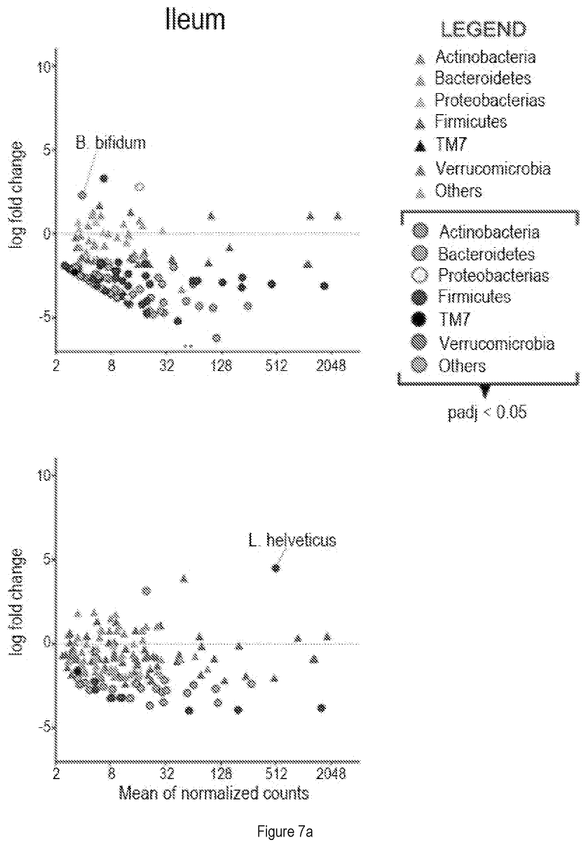

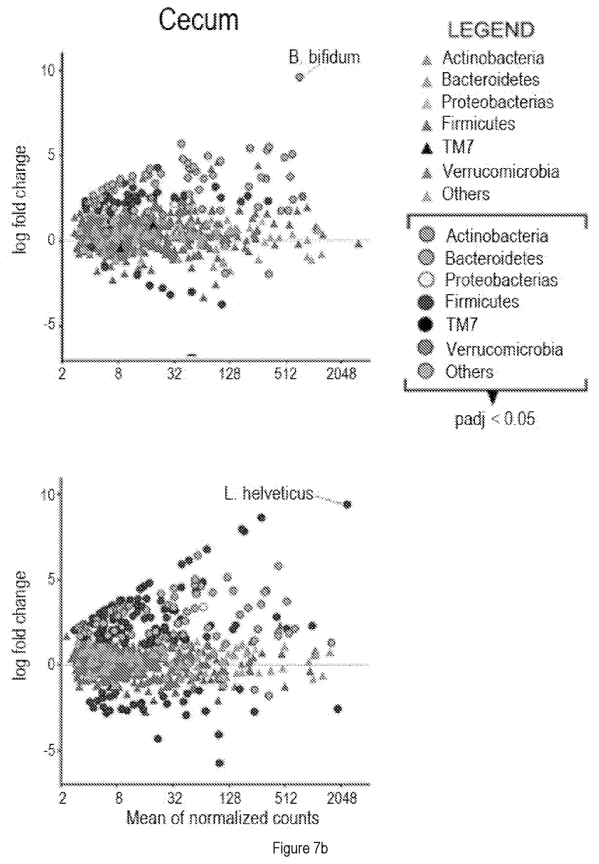

In particular, it was found that the abundance of normalized OTU was mostly decreased in the ileum and increased in the cecum and in the colon by both L. helveticus MIMLh5 and B. bifidum MIMBb23sg DSM 32708 (FIGS. 7a, 7b and 7c). More specifically, 82 OTUs were significantly reduced in the ileum of mice treated with B. bifidum MIMBb23sg DSM 32708 compared to the mice treated with PBS (FIG. 7a), mostly belonging to the phylum Bacteroidetes S24-7 and the phylum Firmicutes.

As concerns the Bifidobacterium bifidum strain, only three OTUs were overrepresented, including the OTU putatively corresponding to the strain B. bifidum MIMBb23sg DSM 32708 (Tables 3A-3B-3C). On the contrary, only 32 OTUs were significantly modified by the strain L. helveticus MIMLh5 in the ileum; specifically, 22 OTUs of Lactobacillus S24-7 and 7 decreased, while only two increased, including the OTU putatively corresponding to the strain L. helveticus MIMLh5 strain (Tables 4A-4B-4C).

In the cecum and in the colon, B. bifidum MIMBb23sg DSM 32708, respectively, significantly increased 101 and 59 OTUs and decreased 8 and 15 OTUs, while the strain MIMLh5, respectively, significantly increased 178 and 112 OTUs and decreased 32 and 13 OTUs (FIGS. 7b and 7c; Tables 3A-3B-3C, Tables 4A-4B-4C). Above all, numerous OTUs were overrepresented in the cecum and in the colon of mice treated with bacteria belonging to the family S24-7, ten of which had also decreased in the ileum of mice treated with MIMBb23sg DSM 32708 (Tables 3A-3B-3C), suggesting a possible relocation of these bacteria from the ileum to more distal intestinal regions after administration of B. bifidum by means of a gastric probe. A significant modulation of numerous OTUs of Clostridiales was also observed in the cecum and in the colon of mice treated with bacteria (Tables 3A-3B-3C, Tables 4A-4B-4C). Furthermore, several OTUs of Lactobacillus were modulated in the cecum and in the colon from the administration of L. helveticus MIMLh5 by means of gastric probe, while two OTUs ascribed to the species Akkermansia muciniphila had increased in mice treated with B. bifidum MIMBb23sg (Tables 3A-3B-3C, TABLES 4A-4B-4C).

Overall, these data demonstrate that, after administration in mice, B. bifidum MIMBb23sg DSM 32708 and L. helveticus MIMLh5 can modulate the composition of the intestinal microbiota, especially in terms of a decrease in the ileum and an increase of the abundance of numerous bacterial OTUs in the cecum and in the colon. In addition, the strain MIMBb23sg DSM 32708 has a higher impact than L. helveticus MIMLh5 on the ileum microbiota, while the strain L. helveticus MIMLh5 had a greater influence on the cecum microbiota than B. bifidum MIMBb23sg DSM 32708.

(XI) B. bifidum MIMBb23sg DSM 32708 has a Greater Influence on the Gene Expression of the Serotoninergic Pathway in the Intestine of Mice with Respect to L. helveticus MIMLh5.

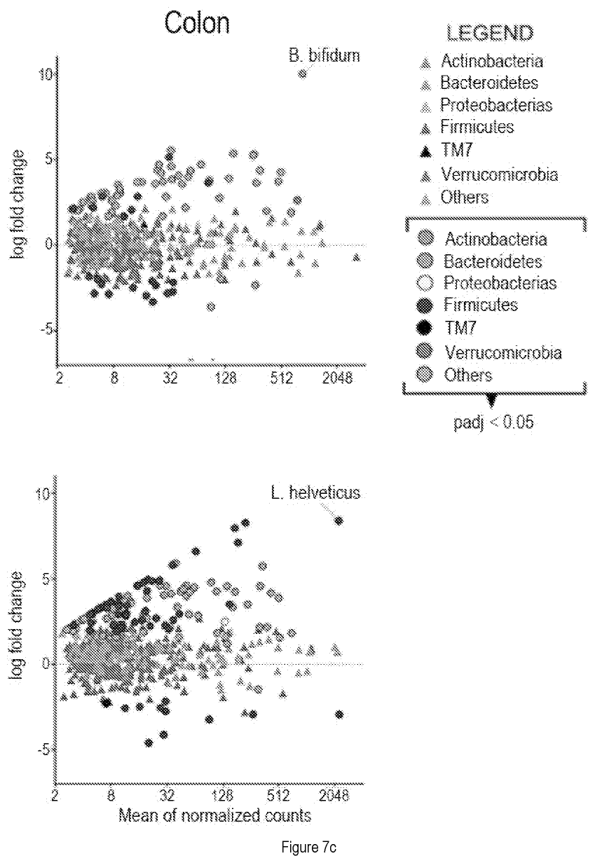

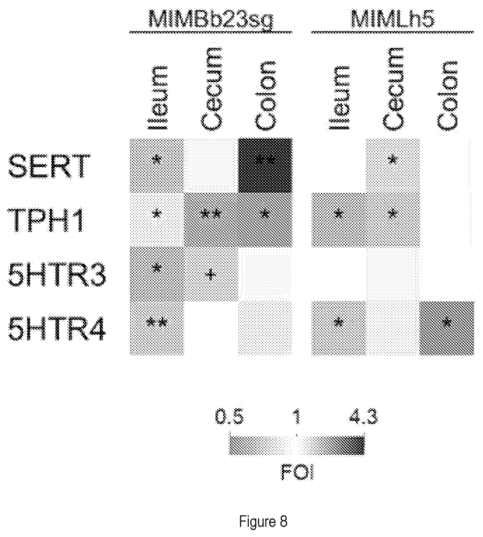

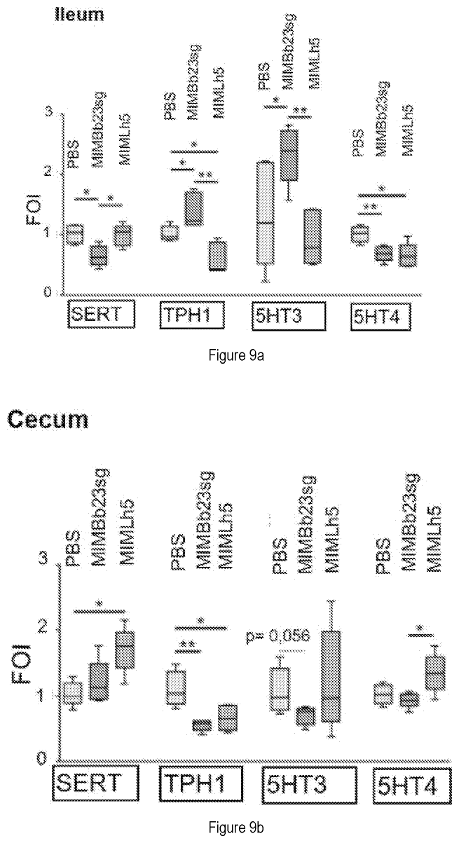

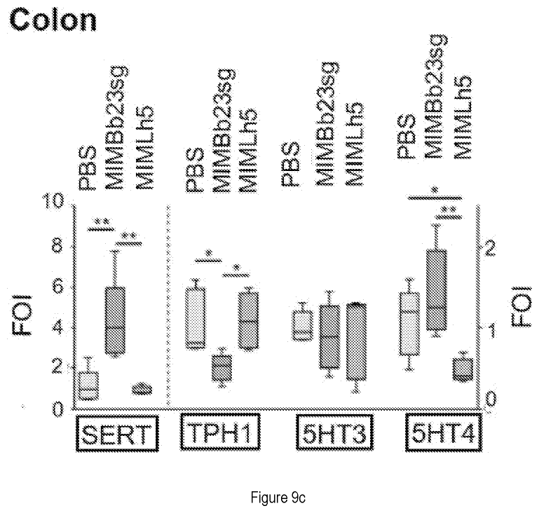

It was hypothesised that a change in intestinal motility may have contributed to the modifications observed in the microbiota and, in particular, to the reduction of the bacterial load in the ileum with the administration of the bacteria. To test this hypothesis, the expression of the genes involved in the intestinal metabolism of serotonin was evaluated. RT-qPCR experiments showed that both strains affected the transcription of the genes involved in the serotonin metabolism (FIG. 8). Nevertheless, B. bifidum MIMBb23 DSM 32708 had a much more evident effect on serotonergic gene expression with respect to L. helveticus MIMLh5, both in the ileum and in the colon (FIG. 8 and FIGS. 9a, 9b and 9c). In particular, the administration of the strain L. helveticus MIMLh5 increased the expression of the gene encoding the receptor for serotonin reuptake (the selective-sodium and chloride-coupled serotonin transporter; SERT) in the cecum, whereas the transcription of tryptophan hydroxylase (TPH1) involved in serotonin synthesis was reduced in the ileum and in the cecum; the expression of the 5HTR4 serotonin receptor was reduced in the ileum and in the colon, whereas the expression of the 5HTR3 receptor was not affected. In contrast, in mice treated with B. bifidum MIMBb23sg DSM 32708 by means of gastric probe, opposite modulation of the expression of the genes involved in the synthesis (TPH1) and in the reuptake (SERT) of serotonin between the ileum and the colon was observed (FIG. 8 and FIGS. 9a and 9c). In fact, while mice treated with B. bifidum MIMBb23sg DSM 32708 by means of gastric probe in the ileum showed an increase in the expression of TPH1 and a reduction in the expression of SERT with respect to control mice, in contrast, the expression of TPH1 was decreased and that of the SERT increased in the colon of mice treated with B. bifidum MIMBb23sg DSM 32708 with respect to mice treated with PBS and L. helveticus MIMLh5.

Significant serotonin 5HTR3 receptor induction in the ileum and decreased expression of 5HTR4 and 5HTR3 in the ileum and cecum, respectively, was also observed.

Overall, these data indicate that L. helveticus MIMLh5 exerts a modest modulation of the expression of the serotonin metabolism genes with respect to B. bifidum MIMBb23sg DSM 32708 and that B. bifidum MIMBb23sg DSM 32708 influences the expression of the main gene involved in the synthesis of serotonin and of the gene encoding the serotonin reuptake protein in the opposite way between the ileum and the colon, suggesting a potential propulsive effect toward the distal part of the intestine.

(XII) The Main Effects on the Intestinal Immune System of Mice are Exercised by B. bifidum MIMBb23sg DSM 32708 in the Ileum.

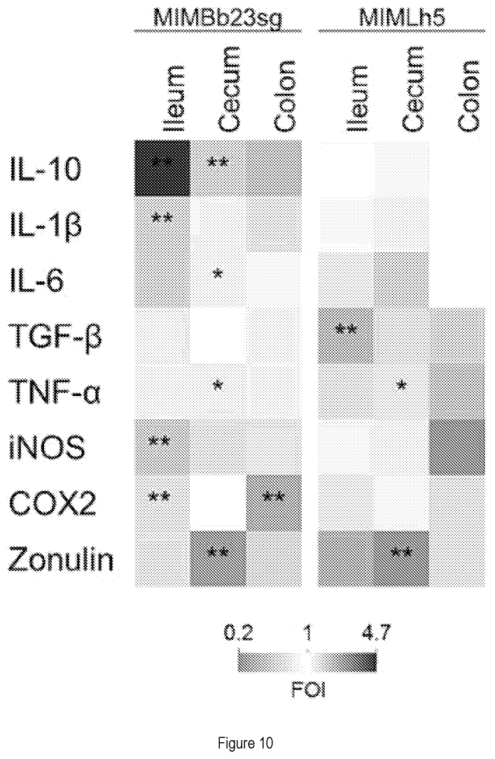

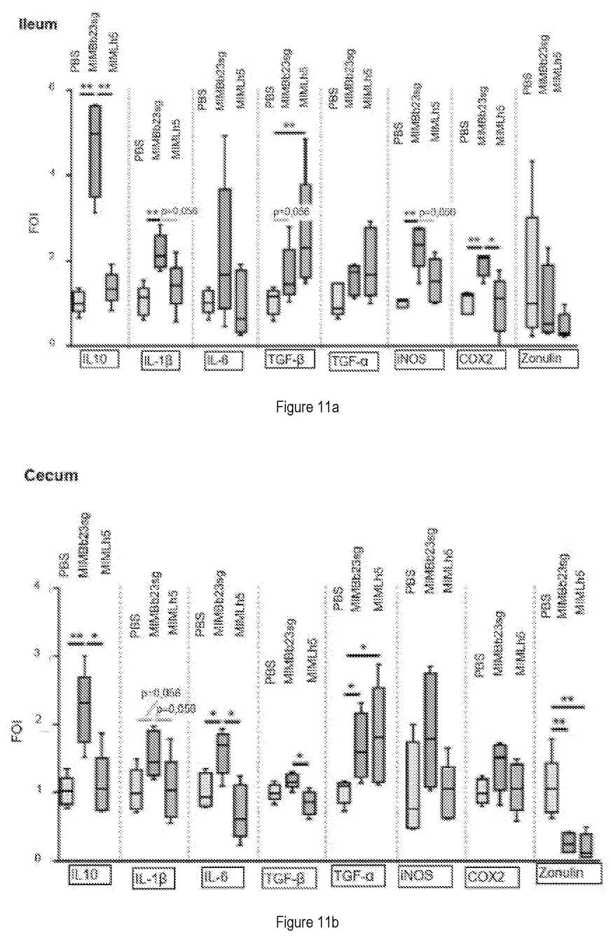

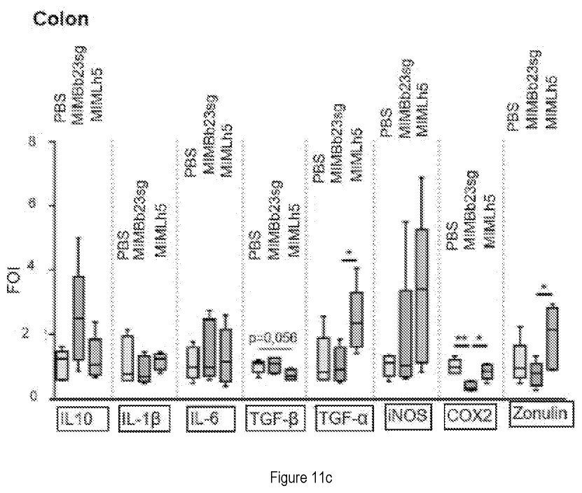

Since intestinal microbiota and the serotoninergic pathway are associated with overall intestinal homeostasis, the immune response and intestinal permeability were also evaluated by performing a targeted RT-qPCR analysis on the genes of different cytokines, cyclooxygenase 2 (COX-2) and zonulin. Similarly to the analysis of the gene expression of serotoninergic metabolism, it was observed that the strain B. bifidum MIMBb23sg DSM 32708 modulates the transcription of immune system genes to a greater extent with respect to L. helveticus MIMLh5. In fact, the administration of B. bifidum MIMBb23sg DSM 32708 induced the expression of several genes involved in innate immunity, especially in the ileum. Specifically, IL-10 was the most increased gene, with a relative FOI of 4.7 (p<0.01) in the ileum. A significant IL-10 induction in the cecum (FOI=2.2; p<0.01) and a tendency increase in the colon (FOI=2.5; p=0.095) was also observed (FIG. 10; FIGS. 11a, 11b, 11c). In addition, in the ileum of mice treated with B. bifidum MIMBb23sg DSM 32708 by means of gastric probe an increase in the expression of the genes encoding iNOS (FOI=2,3; p<0.01), IL-1ß (FOI=2.2; p<0.01) and COX-2 (FOI=1.9; p<0.01) was observed, while a modest but significant induction of TNF-α (FOI=1.7; p<0.05) and IL-6 (FOI=1.6; p<0.05) was observed in the ileum. In the colon, the only significant difference in gene expression was the reduction of COX-2 (FOI=0.4; p<0.05). Lastly, the zonulin gene was modulated exclusively in the cecum, where its expression was reduced in mice treated with B. bifidum MIMBb23sg DSM 32708 (FOI=0.3; p<0.01) and L. helveticus MIMLh5 (FOI=0.2; p<0.01) (FIG. 10; FIG. 11b).

Overall, these results demonstrate that the administration of B. bifidum MIMBb23sg DSM 32708 affected the expression of various genes involved in immune responses in the gut of mice and, in particular, in the ileum, while L. helveticus MIMLh5 had only a limited effect. In particular, the strain B. bifidum MIMBb23sg DSM 32708 induced the expression of IL-10 regulatory cytokine in the ileum and reduced cyclooxygenase (COX-2) in the colon, suggesting that this bacterium can potentially perform an anti-inflammatory/regulatory activity in the intestine.

(XIII) In Vitro Evaluation of the Ability of the Strain B. bifidum MIMBb23sg DSM 32708 to Reduce the Release of IL-8 (Pro-Inflammatory Marker).

In the in vitro test in question, the ability of the bacterial strain of the invention (B. bifidum MIMBb23sg DSM 32708) to modulate the inflammatory response in intestinal cell lines (HT-29), both under baseline conditions and under inflammatory conditions, was evaluated by reading the change in the levels of interleukin-8 (in short, IL-8; pro-inflammatory cytokine).

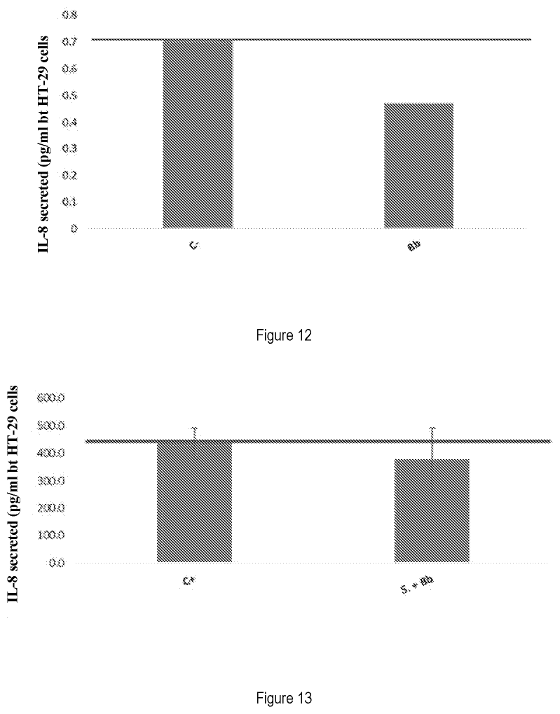

The results (FIGS. 12 and 13) show a positive downregulation trend in the release of IL-8 by the intestinal cell lines following treatment with the strain of the invention (B. bifidum MIMBb23sg DSM 32708) under an inflammatory condition.

In Vitro Test Conduction METHOD:

In short: after 2 hours of exposure of the HT-2 9 cell line to the probiotic bacterial strain of the invention (B. bifidum MIMBb23sg DSM 32708), the probiotic strain was removed and the cell line was placed in a fresh culture medium and incubated another 24 hours. The quantification of IL-8 on the cell line supernatant was carried out subsequently. The test was carried out both under baseline conditions and under inflammatory conditions (the inflammatory stimulus is given by exposure to Salmonella).

Test Under Baseline Conditions:

The HT-29 line is a eukaryotic cell line derived from colon adenocarcinoma. The cell line was cultured until the confluent monolayer was reached in DMEM High Glucose (Dulbecco's Modified Eagle Medium) medium+10% inactivated fetal bovine serum, 2 mm L-glutamine and 50 μg/ml gentamicin at 37° C. in the presence of 5% of CO₂ in the appropriate incubator. The monolayer was trypsinized, the cells were counted by the hemocytometer and 1 ml of the cell suspension diluted to the concentration of 2.5×10⁵ cells/ml was seeded in a 24-well plate and incubated for about 48 hours until confluence. Once confluence was reached, the cells were washed in HBSS and left in DMEM medium without antibiotic and FBS for 2 hours. At the end of this period, the cells were placed at contact with the probiotic strain (MOI 1: 100). At the end of the co-incubation period, the cells were washed twice with HBSS and left for another 24 hours in DMEM without FBS. After this further incubation, the cell supernatant was recovered, centrifuged and used for the subsequent quantification of IL-8 by means of ELISA kit.

Test Under Inflammatory Conditions:

HT-29 cells were seeded as described in the previous paragraph (test under baseline conditions) and, once confluence was reached, they were washed twice with HBSS and left for 2 hours at 37° C. in medium without FBS and without antibiotic. After 2 hours of incubation, an inflammatory condition was induced by challenging HT-29 cells for 2 hours with the pathogenic strain Salmonella enterica Abony NCTC6017. The cells were subsequently washed twice with HBSS and co-incubated with the probiotic strain (MOI 1: 100). At the end of the co-incubation period, the cells were washed twice with HBSS and left for another 24 hours in DMEM without FBS. After this further incubation, the cell supernatant was recovered, centrifuged and used for the subsequent quantification of IL-8 by means of ELISA kit.

MATERIAL used in the in vitro test and abbreviations used in FIGS. 12-13:
Negative control (C−): HT-29+DMEM
Positive control (C+): HT-29+Salmonella enterica Abony NCTC6017
Probiotic strain (Bb): −29+B. bifidum MIMBb23SG DSM 32708

### DISCUSSION OF THE RESULTS

The results reported in the experimental part of the present invention allow the understanding of the mechanisms that support the beneficial properties of the human intestinal microorganism B. bifidum MIMBb23sg DSM 32708. The bacterial strain B. bifidum MIMBb23sg DSM 32708 influences the health of the host through different mechanisms, which can be classified into two main classes: (i) interaction with the intestinal microbial ecology and (ii) modulation of the host's intestinal mucosal metabolism, especially in terms of immunomodulation. In the present study, both aspects were evaluated using a murine model. The initial step of this in vivo study analysed the impact of the administration of B. bifidum MIMBb23sg DSM 32708 on the microbiota of three different intestinal regions. Both the bacterial strain B. bifidum MIMBb23sg DSM 32708 and the control strain L. helveticus MIMLh5 significantly influenced the microbiota of the three enteric sites subject of evaluation, both in terms of composition and total bacterial load. In particular, it was observed that the bacterial cell concentration in the ileum was significantly lower in mice treated with the probiotic with respect to the controls. The amount of bacterial cells in the small intestine must be constantly controlled by the host and thus the excessive expansion thereof induces potentially harmful consequences which are collectively referred to as small intestine bacterial overgrowth (SIBO) syndrome. A recent meta-analysis of 18 clinical trials revealed that probiotic integration can be effective in the treatment of SIBO, reducing the production of H₂ and abdominal pain. Thus, it is possible to contain the bacterial concentration in the small intestine by taking probiotics, which are likely to be due to direct inhibition of bacterial proliferation and/or, alternatively, due to stimulation of small intestine motility. Without ruling out the first hypothesis, the data reported herein provide elements to corroborate the latter, in particular for B. bifidum MIMBb23sg DSM 32708. It was initially observed that besides being reduced in the ileum the total bacterial load of mice treated with these bacteria by means of gastric probe was greater in the distal enteric sites, in particular in the group of mice treated with L. helveticus MIMLh5. Furthermore, several taxonomic units whose abundance was significantly reduced in the ileum were significantly increased in the cecum and in the colon. Lastly, and even more importantly, modulation of serotonergic gene expression toward greater motility in the ileum was observed. In particular, the five-day daily administration of B. bifidum MIMBb23sg DSM 32708 induced a (i) significant expression of the tryptophan hydroxylase-1 gene, TPH1, which represents the limiting enzyme in serotonin biosynthesis, and (ii) the reduction of the gene encoding the serotonin transporter SERT reuptake, thus potentially inducing a higher availability of serotonin in the ileum and, therefore, enhancement of the peristalsis.

The publication of the book “The second Brain” (Gershon, 1998) led to the hypothesis that besides the immune system, the microbiota and the mucosal barrier, a better understanding of the interaction mechanisms between the strain under study used to treat IBS and the host could be achieved by taking into account the enteric nervous system (ENS) and, in particular, serotonergic metabolism. The enteric nervous system (ENS) is defined as an independent neuroendocrine organ governed by the hormone and serotonin neurotransmitter (5-hydroxytryptamine; 5-HT).

Therefore, the present study was conducted to test this hypothesis, collecting data corroborating the hypothesis that the interaction between B. bifidum MIMBb23sg DSM 32708 and the host is also significantly mediated by its ability to modulate the metabolism of 5-HT, obtaining an explanation for the efficacy of this bacterium in the treatment of functional intestinal disorders such as the IBS.

1. A bacterial strain belonging to the species Bifidobacterium bifidum identified as Bifidobacterium bifidum MIMBb23sg=BbfIBS01, wherein said bacterial strain was deposited at Deutsche Sammlung von Mikroorganismen und Zellkulturen GmbH (DSMZ) under deposit number DSM 32708 on 4 Dec. 2017 by Sofar S.p.A.

2. The bacterial strain according to claim 1 for use as a medicament.

3. The bacterial strain according to claim 1 for use in a method for the preventive or curative treatment of gastrointestinal diseases, disorders or symptoms, preferably functional gastrointestinal disorders or inflammatory gastrointestinal disorders.

4. The bacterial strain for use according to claim 3, wherein said strain is for use in a method for preventive and/or curative treatment of functional gastrointestinal disorders selected from: irritable bowel syndrome (IBS), dyspepsia, pyrosis, oesophagus, stomach and duodenum disorders, small intestinal bacterial overgrowth (SIBO), disorders with sub-inflammatory conditions, preferably wherein said sub-inflammatory disorders are manifested in an elderly subject or celiac subject, or in a person suffering from diverticular disease.

5. The bacterial strain for use according to claim 3, wherein said strain is for use in a method for preventive and/or curative treatment of inflammatory gastrointestinal disorders or symptoms selected from: Crohn's disease, ulcerative colitis, microscopic colitis, Helicobacter pylori, peptic or gastric ulcer, duodenal ulcer, celiac disease, diverticular disease and diverticulitis.

6. A composition comprising:
a mixture comprising or, alternatively, consisting of the bacterial strain Bifidobacterium bifidum MIMBb23sg=BbfIBS01 DSM 32708 according to claim 1,
and, optionally, said composition comprises at least one food grade or pharmaceutical additive and/or excipient.

7. The composition according to claim 6, wherein said mixture comprises the bacterial strain B. bifidum MIMBb23sg=BbfIBS01 DSM 32708 at a concentration comprised in the range from 1×10⁶ CFU to 1×10¹² CFU, preferably from 1×10⁷ CFU to 1×10¹¹ CFU, more preferably from 1×10⁸ CFU to 1×10¹⁰ CFU, with respect to a daily intake.

8. The composition according to claim 6 for use as a medicament.

9. The composition according to claim 6 for use in a method for the preventive or curative treatment of gastrointestinal diseases, disorders or symptoms, preferably functional gastrointestinal disorders or inflammatory gastrointestinal disorders.

10. The composition for use according to claim 9, wherein said composition is for use in a method for preventive and/or curative treatment of functional gastrointestinal disorders selected from: irritable bowel syndrome (IBS), dyspepsia, pyrosis, oesophagus, stomach and duodenum disorders, small intestinal bacterial overgrowth (SIBO), disorders with sub-inflammatory conditions, in an elderly subject or in a celiac subject, or in the diverticular disease.

11. The composition for use according to claim 9, wherein said composition is for use in a method for the preventive and/or curative treatment of inflammatory gastrointestinal disorders or symptoms selected from: Crohn's disease, ulcerative colitis, microscopic colitis, Helicobacter pylori, peptic or gastric ulcer, duodenal ulcer, celiac disease, diverticular disease and diverticulitis.

12. The bacterial strain for use according to claim 2, wherein said bacterial strain or said composition is for use in a method for the preventive and/or curative treatment of anxiety and/or depression or related symptoms.

13. A method of treatment comprising administering a composition comprising the bacterial strain of claim 1 to a subject in need thereof.

14. The method of claim 13, wherein the subject has a gastrointestinal disease, disorder or symptom.

15. The method of claim 13, wherein the subject has irritable bowel syndrome (IBS), dyspepsia, pyrosis, oesophagus, stomach and duodenum disorders, small intestinal bacterial overgrowth (SIBO), disorders with sub-inflammatory conditions,

16. The method of claim 15, wherein the subject is elderly, has celiac, or suffers from diverticular disease.

17. The method of claim 13, wherein the patient has Crohn's disease, ulcerative colitis, microscopic colitis, Helicobacter pylori, peptic or gastric ulcer, duodenal ulcer, celiac disease, diverticular disease or diverticulitis.

18. The method of claim 13, wherein the composition comprises the bacterial strain B. bifidum MIMBb23sg=BbfIBS01 DSM 32708 at a concentration comprised in the range from 1×10⁶ CFU to 1×10¹² CFU, with respect to a daily intake.

19. The method of claim 13, wherein the composition further comprises a pharmaceutically acceptable excipient.

20. The method of claim 13, wherein the composition is administered rectally or orally.


SEQUENCE LISTING (18 sequences)

SEQ ID NO: 1 - DNA, 21 bp, Artificial Sequence
  GAPDH F
atgaccacagtccatgccatc

SEQ ID NO: 2 - DNA, 20 bp, Artificial Sequence
  GAPDH R
ggtcctcagtgtagcccaag

SEQ ID NO: 3 - DNA, 19 bp, Artificial Sequence
  SERT F
caaaaccaagaaccaagag

SEQ ID NO: 4 - DNA, 19 bp, Artificial Sequence
  SERT R
catagccaatgacagacag

SEQ ID NO: 5 - DNA, 21 bp, Artificial Sequence
  5-HTR3 F
gtgataagcctcgctgagacc

SEQ ID NO: 6 - DNA, 19 bp, Artificial Sequence
  5-HTR3 R
cgcatctcatcccgcttct

SEQ ID NO: 7 - DNA, 18 bp, Artificial Sequence
  5-HTR4 F
gatgccctttggtgccat

SEQ ID NO: 8 - DNA, 21 bp, Artificial Sequence
  5-HTR4 R
cagcagatggcgtaatacctg

SEQ ID NO: 9 - DNA, 21 bp, Artificial Sequence
  TPH-1 F
atgagagaatttgccaagacc

SEQ ID NO: 10 - DNA, 21 bp, Artificial Sequence
  TPH-1 R
cgtgaactatatttccctcag

SEQ ID NO: 11 - DNA, 16 bp, Artificial Sequence
  ZONU F
gctatgtggattggtt

SEQ ID NO: 12 - DNA, 16 bp, Artificial Sequence
  ZONU R
tccatagagcgatgat

SEQ ID NO: 13 - DNA, 18 bp, Artificial Sequence
  BopA F
gtgttcccaccaactaca

SEQ ID NO: 14 - DNA, 18 bp, Artificial Sequence
  BopA R
gatctggtcgtaaccagt

SEQ ID NO: 15 - DNA, 22 bp, Artificial Sequence
  HELV F
aaacgggcattttgtgggctat

SEQ ID NO: 16 - DNA, 22 bp, Artificial Sequence
  HELV R
gaagcttaaggttgaagatgcc

SEQ ID NO: 17 - DNA, 17 bp, Artificial Sequence
  357 F
cctacgggaggcagcag

SEQ ID NO: 18 - DNA, 20 bp, Artificial Sequence
  907 R
ccgtcaattcctttgagttt